(19) United States
(12) Patent Application Publication
(10) Pub. No.: US 2005/0233453 A1
(43) Pub. Date: Oct. 20, 2005

(54) 6-O-sulfated N-acetylheparosan and hematopoietic stem cell growth auxiliary agent

(75) Inventors: Yutaka Kariya, Higashiyamato-shi (JP); Mamoru Kyogashima, Higashiyamato-shi (JP); Kiyoshi Suzuki, Higashiyamato-shi (JP); Yousuke Yasuda, Higashiyamato-shi (JP)

Correspondence Address: SUGHRUE MION, PLLC, 2100 PENNSYLVANIA AVENUE, N.W., SUITE 800, WASHINGTON, DC, 20037, US

(73) Assignee: SEIKAGAKU CORPORATION

(21) Appl. No.: 11/098,417

(22) Filed: Apr. 5, 2005

Related U.S. Application Data
(60) Provisional application No. 60/559,030, filed on Apr. 5, 2004.

(51) Int. Cl. C12N 5/08; C08B 37/10

(52) U.S. Cl. 435/372; 435/404; 536/21

(57) ABSTRACT

A 6-O-sulfated N-acetylheparosan in which a primary hydroxyl group of N-acetylglucosamine constituting N-acetylheparosan is sulfated; a hematopoietic stem cell growth auxiliary agent, which has hematopoietic stem cell growth-accelerating activity and comprises the 6-O-sulfated-N-acetylheparosan as an active ingredient; a medium for a hematopoietic stem cell, which comprises the hematopoietic stem cell growth auxiliary agent and an other medium component necessary for culturing the hematopoietic stem cell; a method for culturing a hematopoietic stem cell, which comprises culturing the hematopoietic stem cell in the presence of the hematopoietic stem cell growth auxiliary agent or in the medium for a hematopoietic stem cell; and a method for treating a blood disease, which comprises transplanting a hematopoietic stem cell cultured by the above method to bone marrow of a mammal.

### BACKGROUND OF THE INVENTION

1. Field of the Invention

The present invention relates to a 6-O-sulfated-N-acetylheparosan wherein a primary hydroxyl group of N-acetylglucosamine constituting N-acetylheparosan is sulfated. Moreover, the present invention relates to a hematopoietic stem cell growth auxiliary agent comprising the 6-O-sulfated-N-acetylheparosan as an active ingredient, and a process for culturing hematopoietic stem cells using the same.

2. Brief Description of the Background Art

Currently, necessity of regenerative medicine to treat diseases by transplanting stem cells derived from human has been loudly pointed out. As one novel treating method for blood-related diseases such as childhood leukemia, one field of regenerative medicine to administer hematopoietic stem cells derived from cord blood is in the progress of establishing. In this case, the larger the number of the hematopoietic stem cells to be administered is, the higher the healing effect of leukemia is. For example, it is of great significance to grow hematopoietic stem cells wherein coincidence of three or more loci among five loci of HLA antigen is achieved with maintaining its juvenilization level (expansion) after the selection of the hematopoietic stem cells in order to reduce rejection reactions.

It is known that N-desulfated-N-reacetylated heparin obtained by successive N-desulfation and N-reacetylation of heparin is very similar to the structure of a heparan sulfate chain being a constitutive component of heparan sulfate proteoglycan localized on the surface of stromal cells in the niche of the spinal cord and the heparan sulfate chain contributes stabilization of hematopoietic stem cells as a very good reservoir of the hematopoietic stem cells in the niche of the spinal cord (Blood, 92, 4641-4651 (1998)).

Moreover, at culturing hematopoietic stem cells ex vivo, it is proposed to culture them in a medium to which N-desulfated-N-reacetylated heparin is added (Blood, 95, 147-155 (2000)). Furthermore, from recent research, it is found that the growth-accelerating ability of heparan sulfate having a structure similar to N-desulfated-N-reacetylated heparin is based on the affinity to MIP-1α which is one kind of cytokine (Blood, 101, 2243-2245 (2003)).

However, since N-desulfated-N-reacetylated heparin is produced by using heparin isolated and purified from organs of animals such as cattle and swine as starting materials, there is a drawback that a risk of contamination with pathogenic viruses and prion proteins is unavoidable.

Heparin or heparin analogs independent of animal organs are highly useful as mentioned above and their industrial production has been extensively studied.

For example, there are mentioned N-acetylheparosan and the like, which are considered as heparin precursors. N-Acetylheparosan is a glycosaminoglycan which is characterized by a repeating structure of the disaccharide unit of glucuronic acid and N-acetylglucosamine, is a capsular polysaccharide produced by a certain strain of Escherichia coli, and is called K5 antigen (Eur. J Chem., 116, 359-364 (1981)).

Moreover, derivatives of N-acetylheparosan include N,O-sulfated-heparosan obtained by chemical sulfation (JP-A-5-271305), O-sulfated-K5 polysaccharide obtained by direct sulfation of K5 polysaccharide (JP-T-2001-510502, WO98/34958), sulfaminoheparosan sulfate obtained by deacetylation and sulfation (JP-T-2000-517328, WO98/09636), and the like.

However, there have not been known 6-O-sulfated-N-acetylheparosan itself and the fact that it has a hematopoietic stem cell growth-accelerating activity.

### SUMMARY OF THE INVENTION

Accordingly, an object of the present invention is to provide a heparan sulfate analog having an excellent hematopoietic stem cell growth-accelerating activity and having no possibility of contamination with pathogenic viruses and prion proteins, the analog being capable of replacing N-desulfated-N-reacetylated heparin which is known to have such the activity.

In consideration of the above circumstances, as a result of extensive studies, the present inventors have succeeded in the synthesis of 6-O-sulfated-N-acetylheparosan wherein a primary hydroxyl group of N-acetylglucosamine constituting N-acetylheparosan is sulfated. Furthermore, they have found that the substance has an excellent effect of accelerating hematopoietic stem cell growth and thus have accomplished the present invention.

Namely, the present invention relates to the followings.

(1) A 6-O-sulfated N-acetylheparosan in which a primary hydroxyl group of N-acetylglucosamine constituting N-acetylheparosan is sulfated.

(2) The 6-O-sulfated-N-acetylheparosan according to (1), wherein the content of 2-acetamido-2-deoxy-4-O-(4-deoxy-α-L-threo-hex-4-enopiranosyluronic acid)-6-O-sulfo-D-glucose represented by ΔDiHS-6S is from 30 to 70% by mole in an unsaturated disaccharide obtainable by a disaccharide composition analysis in combination of degradation by a glycosaminoglycan-degrading enzyme with analysis on high performance liquid chromatography.

(3) The 6-O-sulfated-N-acetylheparosan according to (1) or (2), wherein the content of 2-acetamido-2-deoxy-4-O-(4-deoxy-α-L-threo-hex-4-enopiranosyluronic acid)-D-glucose represented by ΔDiHS-0S is from 70 to 30% by mole in an unsaturated disaccharide obtainable by a disaccharide composition analysis in combination of degradation by a glycosaminoglycan-degrading enzyme with analysis on high performance liquid chromatography.

(4) A hematopoietic stem cell growth auxiliary agent, which has hematopoietic stem cell growth-accelerating activity and comprises the 6-O-sulfated-N-acetylheparosan according to any one of (1) to (3) as an active ingredient.

(5) The hematopoietic stem cell growth auxiliary agent according to (4), wherein the hematopoietic stem cell is a hematopoietic stem cell derived from human.

(6) The hematopoietic stem cell growth auxiliary agent according to (4) or (5), wherein the hematopoietic stem cell is a hematopoietic stem cell derived from bone marrow, peripheral blood or cord blood.

(7) The hematopoietic stem cell growth auxiliary agent according to any one of (4) to (6), which is used for culturing cells in vitro or ex vivo.

(8) The hematopoietic stem cell growth auxiliary agent according to any one of (4) to (7), which is used for accelerating growth of a hematopoietic stem cell for transplant to a living body.

(9) A medium for culturing a hematopoietic stem cell, which comprises the hematopoietic stem cell growth auxiliary agent according to any one of (4) to (8) and an other medium component necessary for culturing the hematopoietic stem cell.

(10) The medium for culturing a hematopoietic stem cell according to (9), wherein the other medium component is at least one cytokine selected from the group consisting of interleukin 3, macrophage inflammatory proteins, stem cell factors, and platelet factor 4.

(11) A method for culturing a hematopoietic stem cell, which comprises culturing the hematopoietic stem cell in the presence of the hematopoietic stem cell growth auxiliary agent according to any one of (4) to (8) or in the medium for culturing a hematopoietic stem cell according to (9) or (10).

(12) A method for treating a blood disease, which comprises transplanting a hematopoietic stem cell cultured by the method according to (11) to bone marrow of a mammal.

### BRIEF DESCRIPTION OF THE DRAWINGS

FIG. 1 shows elution profiles of Reaction-A products on an anion exchange chromatography.

FIG. 2 shows gel permeation HPLC analysis patterns of Fractions I and II of Reaction-A and Reaction-B Product.

FIG. 3 shows strong ion exchange HPLC analysis patterns of Fractions I and II of Reaction-A and Reaction-B Product.

### DETAILED DESCRIPTION OF THE INVENTION

The present invention is described below in detail.

The 6-O-sulfated-N-acetylheparosan of the present invention is one wherein a primary hydroxyl group of N-acetylglucosamine constituting N-acetylheparosan is sulfated.

The method for producing the 6-O-sulfated-N-acetylheparosan of the present invention is not particularly limited as far as it specifically sulfates the primary hydroxyl group of N-acetylheparosan, and examples thereof include a method of using a complex of sulfur trioxide with an organic base, such as trimethylamine, triethylamine or pyridine, in an aprotic solvent such as dimethyl sulfoxide (DMSO), dimethylformamide (DMF) or pyridine; a method of using a glycosaminoglycan-6-O-sulfotransferase having an activity to transfer a sulfate group into the primary hydroxyl group of glycosamine of glycosaminoglycan; and the like.

As the N-acetylheparosan to be used as a starting material, use can be made of one obtained by a method of culturing of Escherichia coli having an N-acetylheparosan-producing ability and purification from the culture.

The Escherichia coli having an N-acetylheparosan-producing ability is not particularly limited and examples thereof include K5 strain or strains having substantially the same properties as those of the strain. As the K5 strain, use can be made of type culture strain easily available by those skilled in the art.

When the 6-O-sulfated-N-acetylheparosan thus produced is digested with a glycosaminoglycan-degrading enzyme such as a heparin-degrading enzyme (heparinase, heparitinase I, II) and the composition of unsaturated disaccharides produced is analyzed by high performance liquid chromatography (HPLC), the content of 2-acetamido-2-deoxy-4-O-(4-deoxy-α-L-threo-hex-4-enopiranosyluronic acid)-6-O-sulfo-D-glucose (hereinafter referred to as “ΔDiHS-6S”) is from 30 to 70% by mole and the content of 2-acetamido-2-deoxy-4-O-(4-deoxy-α-L-threo-hex-4-enopiranosyluronic acid)-D-glucose (hereinafter referred to as “ΔDiHS-0S”) is from 70 to 30% by mole.

Moreover, when measured by gel permeation chromatography using HPLC to be mentioned below, the weight-average molecular weight is usually in the range of 35,000 to 450,000, preferably 40,000 to 80,000.

In the present specification, the weight-average molecular weight (Mw) particularly means a molecular weight measured using a gel permeation chromatographic method by BPLC (Biochem. Biophys. Acta., 1117, 60-70 (1992)).

The hematopoietic stem cell growth-accelerating activity of 6-O-sulfated-N-acetylheparosan of the present invention is considered to be higher than that of N-desulfated-N-reacetylated heparin, and the activated partial thromboplastin time (APTT) is considered to be shorter than that of the N-desulfated-N-reacetylated heparin, so that the anticoagulant activity is also considered to be low.

The hematopoietic stem cell growth auxiliary agent of the present invention is suitably used as a growth auxiliary agent having cell growth-accelerating activity in culturing hematopoietic stem cells in vitro or ex vivo.

In this connection, the growth auxiliary agent may contain water, a buffer, and the like in addition to the 6-O-sulfated-N-acetylheparosan.

The hematopoietic stem cells to be used in the method of culturing a hematopoietic stem cell of the present invention is not particularly limited as far as they are derived from a vertebrate and are preferably stem cells derived from bone marrow, peripheral blood, or cord blood of a mammal, particularly human. In the case of using them for treating blood diseases, more preferred are allogenic hematopoietic stem cells which are hematopoietic stem cells of another person and autologous hematopoietic stem cells which are own hematopoietic stem cells.

Moreover, the hematopoietic stem cells cultured and grown in a hematopoietic stem cell-culturing medium containing the hematopoietic stem cell growth auxiliary agent of the present invention can be employed for transplant into a living body for the purpose of treating blood diseases or the like.

The hematopoietic stem cell-culturing medium to be used in the culturing method of the present invention contains the hematopoietic stem cell growth auxiliary agent of the present invention and an other medium component necessary for culturing the cells. As such a medium component, a basal growth medium may be mentioned. Specifically, it is a medium wherein the hematopoietic stem cell growth auxiliary agent of the present invention is added to a basal medium containing components necessary for culturing cells (inorganic salts, carbohydrates, hormones, essential amino acids, and vitamins), for example, Iscove modified Dulbecco medium (IMDM), RPMI, DMEM, Fischer medium, α medium, Leibovitz medium, L-15 medium, NCTC medium, F-12 medium, MEM, or McCoy medium. Moreover, the above medium may further contain interleukin 3, macrophage inflammatory proteins, stem cell factors and platelet factor 4.

The hematopoietic stein cell growth auxiliary agent of the present invention is added to the above medium so that the final concentration of 6-O-sulfated-N-acetylheparosan in the medium is from 1 to 100 μg/ml, preferably from 5 to 20 μg/ml.

The 6-O-sulfated-N-acetylheparosan of the present invention has a hematopoietic stem cell growth-accelerating activity and has no possibility of the contamination with pathogenic viruses and prion proteins, and hence it can provide a hematopoietic stem cell growth accelerator which is low in anticoagulation activity and safe and which can be continuously supplied.

The present invention is described below in more detail based on Examples, but the present invention is not limited thereto.

### EXAMPLE 1

### Synthesis of 6-O-sulfated-N-acetylheparosan

After 218.7 mg of N-acetylheparosan obtained from a culture of Escherichia coli K5 strain was dissolved in 10 ml of distilled water, the solution was passed through an Amberlite IR-120B (manufactured by Organo) column (φ2×15 cm) equilibrated with distilled water and the eluate was fractionated every 3 ml. After measuring pH of 30 fractions finally obtained, acidic fractions were collected and 0.42 ml of n-tributylamine (TBA) corresponding to 3.0 equivalents of total carboxyl groups of N-acetylheparosan was added. By lyophilization of the solution, N-acetylheparosan TBA salt was obtained. In order to carry out a 6-O-sulfation reaction specifically, 25 ml of dimethylformamide (DMF) was mixed with X mg of N-acetylheparosan according to the following two kinds of reactions, followed by stirring under a temperature condition of 50° C. for 4 hours. Furthermore, after Y mg of pyridine-sulfotrioxide complex (Py-SO₃) was added, the reaction was forwarded at 50° C. for 2 hours under a stirring condition. In this connection, the following two kinds of Reactions-A and -B were carried out based on the combination of X and Y.

Reaction-A: X=50.4 mg, Y=83.9 mg (3.1 equivalents per disaccharide unit)

Reaction-B: X=46.5 mg, Y=86.0 mg (3.8 equivalents per disaccharide unit)

Herein, the equivalent is shown assuming that the carboxyl groups of the N-acetylheparosan TBA salt used is quantitatively converted into its n-tributylamine salt and the salt is a lyophilized powder containing 10% moisture.

In order to terminate the reaction, the reaction solution was ice-cooled and 25 ml of distilled water was added, followed by dialysis against running tap water. The retantate of the dialysis was concentrated and then passed through Amberlite IR-120B column (φ2×15 cm). After measuring pH of 30 fractions finally obtained, acidic fractions were collected and adjusted to pH 7.0 with 1N NaOH. The collected fraction was purified successively on a Cellulofine GCL-90 (available from Seikagaku Corporation) column (φ3×120 cm) equilibrated with 0.2M NaCl and then on a Cellulofine GCL-25 (available from Seikagaku Corporation) column (φ3.3×36 cm) equilibrated with distilled water. Thereafter, by lyophilization, the thus obtained powders derived from Reactions-A and -B were obtained in amounts of 42.7 mg and 52.2 mg, respectively.

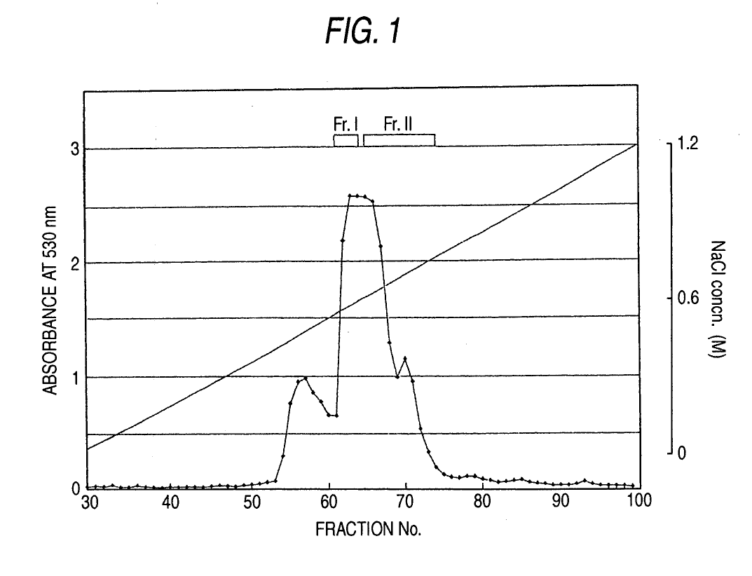

Then, 41.8 mg of the reaction product of Reaction-A was dissolved in 30 ml of 100 mM sodium acetate (pH 5.0) and applied to a Whatman DE52 column (φ2.3×18 cm) equilibrated with the same buffer. After washing with 100 ml of 100 mM sodium acetate (pH 5.0), the column was eluted with a linear increasing gradient of NaCl using 200 ml of the same buffer and 200 ml of 100 mM sodium acetate (pH 5.0) containing 1.2M NaCl. FIG. 1 shows an elution profile in which detected values (OD530) of uronic acid by a carbazole method were plotted. As shown in the figure, Fractions I and II were collected in the order of elution and were desalted and lyophilized separately. The weights of the resulting Fractions I and II were 15.2 mg and 15.4 mg, respectively.

### EXAMPLE 2

### Gel Permeation HPLC of 6-O-sulfated-N-acetylheparosan and Measurement of Molecular Weight Thereof

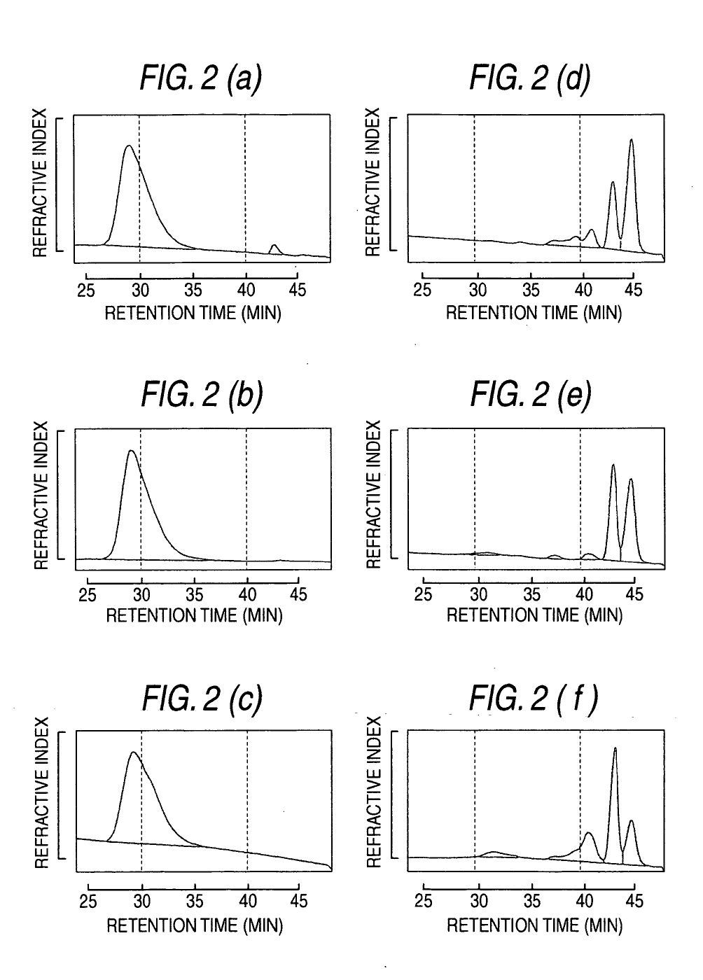

A gel permeation (GPC)-HPLC analysis was carried out by applying 50 μg/5 μl of each of N-acetylheparosan and 6-O-sulfated-N-acetylheparosan to CCPM-type HPLC system (manufactured by TOSOH) in which columns of TSKgel-PW_{XL} 4,000, 3,000, and 2,500 types were serially combined and equilibrated with 0.2M NaCl. The analysis was carried out at 40° C. using a column oven CO-8020 (manufactured by Tosoh), and at 0.6 ml/minute. The detection was performed with refractive index (RI) using a refractive index meter RI-8020 (manufactured by Tosoh). As a result, as shown in FIG. 2, Fraction I derived from Reaction-A (FIG. 2a), Fraction II derived from Reaction-A (FIG. 2b), and Reaction-B Product (FIG. 2c) were eluted at retention times of 29.1 minutes, 29.0 minutes, and 28.9 minutes. As a result of the comparison with a calibration curve using molecular weight specimens, the molecular weights of Fraction I derived from Reaction-A, Fraction II derived from Reaction-A, and Reaction-B Product were calculated as 6.1×10⁴ Da, 6.2×10⁴ Da, and 6.3×10⁴ Da, respectively. FIGS. 2d, 2e, and 2f show patterns of Fraction I derived from Reaction-A, Fraction II derived from Reaction-A, and Reaction-B Product after enzymatic digestion.

### EXAMPLE 3

### Analysis of Unsaturated Disaccharide Composition of 6-O-sulfated-N-acetylheparosan

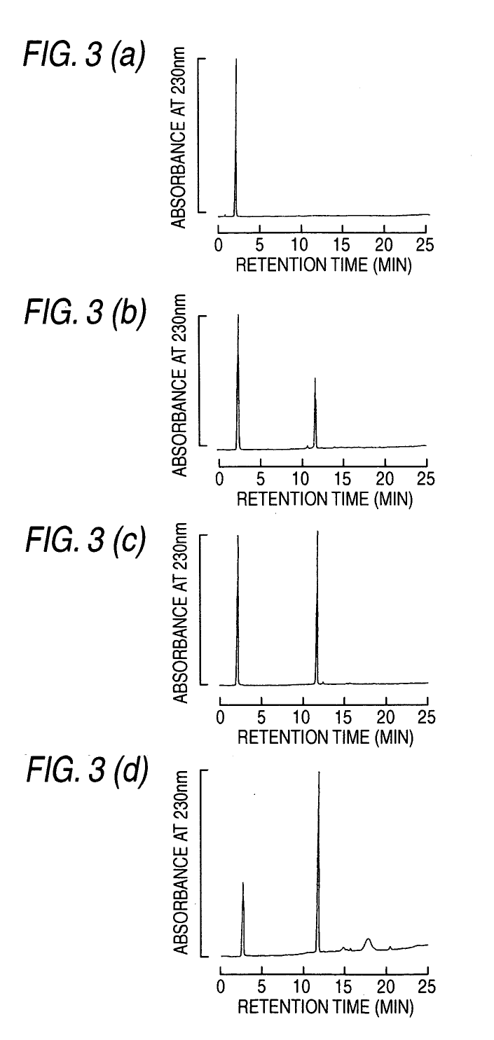

The analysis of the unsaturated disaccharide composition was carried out in accordance with the method of Kariya et al (Comp. Biochem. Physiol., 103B, 473-479 (1992)). The enzymatic digestion was carried out under conditions of 37° C. and 2 hours per 200 μg of each substrate using a mixture of 40 mU of each of heparitinase I, II and heparinase (each manufactured by SEIKAGAKU CORPORATION). As a result of analysis of the enzymatic digestion products on strong ion exchange (SAX)-HPLC (CarboPak PA-1 column, manufactured by Dionex), SAX-HPLC patterns of Fraction I derived from Reaction-A, Fraction II derived from Reaction-A, and Reaction-B Product were obtained as shown in FIG. 3. FIG. 3a is an SAX-IIPLC pattern of the enzymatic digestion product of N-acetylheparosan as a control. FIGS. 3b, 3c, and 3d show SAX-HPLC patterns of that of Fraction I derived from Reaction-A, Fraction II derived from Reaction-A, and Reaction-B Product, respectively. In the pattern of N-acetylheparosan in FIG. 3a, a single peak of ΔDiHS-0S was only observed at a retention time of 2.5 minutes.

On the other hand, in the pattern of Fraction I derived from Reaction-A in FIG. 3b, a peak of ΔDiHS-6S was newly observed at a retention time of 12 minutes in addition to the peak of ΔDiHS-0S at 2.5 minutes. The area ratio of the both peaks: ΔDiHS-0S:ΔDiHS-6S was 2:1. In the pattern of Fraction II derived from Reaction-A in FIG. 3c, the peak of ΔDiHS-0S at 2.5 minutes and the peak of ΔDiHS-6S at 12 minutes were also observed. The area ratio of both peaks: ΔDiHS-0S:ΔDiHS-6S was 1:1. From these results, it was shown that, in Reaction-A, specific sulfation proceeded in about ⅓ to ½ of the primary hydroxyl group of N-acetylglucosamine constituting N-acetylheparosan. In the pattern of Reaction-B Product in FIG. 3d, the peak of ΔDiHS-0S at 2.5 minutes and the peak of ΔDiHS-6S at 12 minutes were observed as main peaks, while a broad minor peak was detected at 17.5 minutes, which was thought to be an incompletely split oligosaccharide. The area ratio of both main peaks: ΔDiHS-0S:ΔDiHS-6S was 1:2. From this result, it was shown that, in Reaction-B, specific sulfation proceeded in about ⅔ of the primary hydroxyl group of N-acetylglucosamine constituting N-acetylheparosan. Namely, in Example 1, it was revealed that the sulfation specific to the primary hydroxyl group proceeded in Reaction-B 4/3 to 2 times more effectively than in Reaction-A. As above, using N-acetylheparosan as a starting material, 6-O-sulfated-N-acetylheparosan having a structure originated in the chemical modification mode designed beforehand was synthesized as a series of derivatives having gradual degree of sulfation.

### EXAMPLE 4

### Measurement of Hematopoietic Stem Cell Growth Activity

In accordance with the method described in Blood, 95, 147-155 (2000), the hematopoietic stem cell growth-accelerating activity of Fraction I derived from Reaction-A, Fraction II derived from Reaction-A, and Reaction-B Product is measured ex vitro. Namely, as a hematopoietic stem cell growth medium, an ISCOVE modified Dulbecco medium (LTBMC medium) containing 12.5% fetal calf serum, 12.5% horse serum, 2 mM L-glutamine, 1000 U/ml penicillin, 100 U/ml streptomycin, and 10⁻⁶ mol/L hydrocortisone is used as a basal medium and, after the addition of IL-3 and MIP-1α to the LTBMC medium, each 6-O-sulfated-N-acetylheparosan (Fraction I derived from Reaction-A, Fraction II derived from Reaction-A, and Reaction-B Product) is added thereto to form a hematopoietic stem cell growth medium to be used. CD34⁺/HLA-DR⁻ cells (hematopoietic stem cells) are added to each well of a 6- or 24-well microtiter plate so as to be 10-14×10³ cells/well and furthermore, the above hematopoietic stem cell growth medium was added in an amount of 3 ml (6 well) or 0.8 ml (24 well) each. Culturing is conducted at 37° C. in 5% CO₂. After 2 to 5 weeks, CD34⁺/HLA-DR⁻ cell forming colonies was taken out of each well and transferred to a methylcellulose medium. The number of cells of the above colony-forming cells (CFC) was measured using a limiting dilution analytical method.

As shown in Example 3, since the primary hydroxyl group of N-acetylglucosamine is sulfated in about 30%, 50%, and 70% of the disaccharide units of Fraction I derived from Reaction-A, Fraction II derived from Reaction-A, and Reaction-B Product, it is considered that all these structures have structures analogous to N-desulfated-N-reacetylated heparin and exhibit a hematopoietic stem cell growth-accelerating activity and the hematopoietic stem cell growth-accelerating activity increases depending on the sulfate content (Fraction I derived from Reaction-A<Fraction II derived from Reaction-A<Reaction-B Product).

### EXAMPLE 5

### Measurement of Activated Partial Thromboplastin Time (APTT)

Hundred μl of plasma obtained by centrifuging at 1000×g for 10 minutes blood sampled from descending aorta of a rat with 1/10 volume of 3.2% citric acid and 100 μl of a different concentration of N-desulfated-N-reacetylated heparin or each 6-O-sulfated-N-acetylheparosan (Fraction I derived from Reaction-A, Fraction II derived from Reaction-A, and Reaction-B Product) were placed in a cup for measurement and the whole was kept at 37° C. for 1 minute. Thereafter, 100 μl of Actin (trade name: Mitsubishi Pharma Corporation) kept at 37° C. beforehand was added thereto, followed by maintenance at the temperature for 2 minutes. Then, 100 μl of a 0.02M calcium chloride solution kept at 37° C. was added and a time required for the occurrence of coagulation from this point was measured by means of an automatic blood coagulation-measuring apparatus (KC-10A; manufactured by Amelung).

As a result, it is considered that APTT at a test substance concentration of 100 μg/ml is 80 seconds or more in the case of N-desulfated-N-reacetylated heparin but is 70 seconds or less in all the cases of the 6-O-sulfated-N-acetylheparosan. That is, it is assumed that anticoagulation activity of 6-O-sulfated-N-acetylheparosan is weaker than that of N-desulfated-N-reacetylated heparin.

While the invention has been described in detail and with reference to specific embodiments thereof, it will be apparent to one skill in the art that various changes and modifications can be made therein without departing from the spirit and scope thereof. All references cited herein are incorporated in their entirety.

This application is U.S. provisional patent application No. 60/559,030 filed on Apr. 5, 2004, the entire contents of which are incorporated hereinto by reference.

1. A 6-O-sulfated N-acetylheparosan in which a primary hydroxyl group of N-acetylglucosamine constituting N-acetylheparosan is sulfated.

2. The 6-O-sulfated-N-acetylheparosan according to claim 1, wherein the content of 2-acetamido-2-deoxy-4-O-(4-deoxy-α-L-threo-hex-4-enopiranosyluronic acid)-6-O-sulfo-D-glucose represented by ΔDiHS-6S is from 30 to 70%o by mole in an unsaturated disaccharide obtainable by a disaccharide composition analysis in combination of degradation by a glycosaminoglycan-degrading enzyme with analysis on high performance liquid chromatography.

3. The 6-O-sulfated-N-acetylheparosan according to claim 1, wherein the content of 2-acetamido-2-deoxy-4-O-(4-deoxy-α-L-threo-hex-4-enopiranosyluronic acid)-D-glucose represented by ΔDiHS-0S is from 70 to 30% by mole in an unsaturated disaccharide obtainable by a disaccharide composition analysis in combination of degradation by a glycosaminoglycan-degrading enzyme with analysis on high performance liquid chromatography.

4. A hematopoietic stem cell growth auxiliary agent, which has hematopoietic stem cell growth-accelerating activity and comprises the 6-O-sulfated-N-acetylheparosan according to claim 1 as an active ingredient.

5. The hematopoietic stem cell growth auxiliary agent according to claim 4, wherein the hematopoietic stem cell is a hematopoietic stem cell derived from human.

6. The hematopoietic stem cell growth auxiliary agent according to claim 4, wherein the hematopoietic stem cell is a hematopoietic stem cell derived from bone marrow, peripheral blood or cord blood.

7. The hematopoietic stem cell growth auxiliary agent according to claim 4, which is used for culturing cells in vitio or ex vivo.

8. The hematopoietic stem cell growth auxiliary agent according to claim 4, which is used for accelerating growth of a hematopoietic stem cell for transplant to a living body.

9. A medium for culturing a hematopoietic stem cell, which comprises the hematopoietic stem cell growth auxiliary agent according to claim 4 and an other medium component necessary for culturing the hematopoietic stem cell.

10. The medium for culturing a hematopoietic stem cell according to claim 9, wherein the other medium component is at least one cytokine selected from the group consisting of interleukin 3, macrophage inflammatory proteins, stem cell factors, and platelet factor 4.

11. A method for culturing a hematopoietic stem cell, which comprises culturing the hematopoietic stem cell,
in the presence of the hematopoietic stem cell growth auxiliary agent according to claim 4 or
in the medium for culturing a hematopoietic stem cell, which comprises the hematopoietic stem cell growth auxiliary agent according to claim 4 and an other medium component necessary for culturing the hematopoietic stem cell.

12. A method for treating a blood disease, which comprises transplanting a hematopoietic stem cell cultured by the method according to claim 11 to bone marrow of a mammal.
